(19) United States
(12) Patent Application Publication
(10) Pub. No.: US 2019/0374108 A1
(43) Pub. Date: Dec. 12, 2019

(54) QUANTUM-DOT SPECTROMETERS FOR USE IN BIOMEDICAL DEVICES AND METHODS OF USE

(71) Applicant: Johnson & Johnson Vision Care, Inc., Jacksonville, FL (US)

(72) Inventors: Frederick A. Frederick A. Flitsch, New Windsor, NY (US); Jorge Gonzalez, Jacksonville, FL (US); Randall B. Pugh, Jacksonville, FL (US)

(21) Appl. No.: 16/215,265

(22) Filed: Dec. 10, 2018

Related U.S. Application Data
(63) Continuation of application No. 14/863,933, filed on Sep. 24, 2015, now abandoned.

(51) Int. Cl. A61B 5/00; A61B 3/10

(52) U.S. Cl. CPC A61B 5/0075; A61B 5/7225; A61B 5/0084; A61B 3/10

(57) ABSTRACT

Device and methods for the incorporation of Quantum-Dots for spectroscopic analysis into biomedical devices are described. In some examples, the Quantum-Dots act as light emitters, light filters or analyte specific dyes. In some examples, a field of use for the apparatus and methods may include any biomedical device or product that benefits from spectroscopic analysis.

### CLAIM OF PRIORITY

This application is a continuation of U.S. patent application Ser. No. 14/863,933 filed Sep. 24, 2015.

### BACKGROUND OF THE INVENTION

### 1. Field of the Invention

Quantum-dot spectrometers for use in biomedical devices are described herein. In some exemplary embodiments, the devices' functionality involves collecting biometric information to perform personalized bioanalysis for the user of the device.

### 2. Discussion of the Related Art

Recently, the number of medical devices and their functionality has begun to rapidly develop. These medical devices may include, for example, implantable pacemakers, electronic pills for monitoring and/or testing a biological function, surgical devices with active components, contact lenses, infusion pumps, and neurostimulators. These devices are often exposed to and interact with biological and chemical systems making the devices optimal tools for collecting, storing, and distributing biometric data.

Some medical devices may include components such as semiconductor devices that perform a variety of functions including biometric collection, and may be incorporated into many biocompatible and/or implantable devices. Such semiconductor components require energy and, thus, energization elements must also be included in such biocompatible devices. The addition of self-contained energy in a biomedical device capable of collecting biometric information would enable the device to perform personalized biometric analysis for the user of the device.

One aspect of biometric information collection has focused on the ability to pair an analyte to a corresponding enzyme such as glucose to glucose oxidase for the detection of glucose in a fluid medium. Another aspect of biometric information collection may focus on the use of light where a light source shines light through a medium which is in turn collected by a detector and analyzed for the amount of light absorbed, similar to a spectrometer. Spectrometers are widely used in physical, chemical, and biological research; however, current micro-spectrometer designs mostly use interference filters and interferometric optics that limit their photon efficiency, resolution, and spectral range. Nevertheless, the miniaturization possible with the development of techniques and reagents that utilize quantum-dots in supporting the acquisition of spectroscopic data may allow for significant advances in the ability of biomedical devices to sense chemical states of their environments.

A quantum-dot (QD) is a nanocrystal commonly made of semiconductor materials. When crystals are “nano-sized” they become small enough to exhibit quantum mechanical properties. Technologies around QDs exploit this quantum mechanical behavior to result in interesting optical properties for the QDs. Therefore, novel devices for biomedical purposes for the use of quantum-dots and for quantum-dot spectrometry may be useful.

### SUMMARY OF THE INVENTION

Accordingly, devices and methods for the use of QDs as emission sources, filters, dyes and as narrow and broadband spectrometers on or in powered biomedical devices may enable the powered biomedical devices to specifically and accurately detect analytes on or in the body of a user. Quantum-dots are extremely small entities that can be manufactured with high levels of consistency and purity. Since the quantum-dot manufacturing process may be tuned to different sizes and materials a nearly arbitrary amount of frequencies may be tuned for the spectral response from a type of QD. As emission sources, therefore, fine line fluorescence sources may be formed from the excitation of QDs with their resultant high yield fluorescence emission. For the use of QDs as filters, a tunable transmission response may be obtained. Therefore, it may be easy to create spectrometers comprising hundreds of unique and tuned spectral filters to create the perspective of broadband spectroscopy. Still further spectral relevance of QDs may arise from the fact that individual QDs may have molecules that bind to the surface and quench their fluorescence. These quenching molecules may be selected and designed to bind to analytes and in so doing decouple from the QD that they are quenching, resulting in sensitive fluorescence probes for analyte study.

One general aspect includes forming a biomedical device including an energization element including a first and second current collector, a cathode an anode and an electrolyte. The biomedical device may also comprise a quantum-dot spectrometer which may include a quantum-dot light emitter, a photodetector, and a means of communicating information from the quantum-dot spectrometer to a user. The quantum-dot spectrometer is powered by the energization element. The biomedical device may also include an insert device. This insert device may contain the energization element and the quantum-dot spectrometer. The insert device creates an encapsulation that isolates the energization elements from the biomedical environment that this biomedical device operates within.

The biomedical device may further comprise a micro-fluidic pump. The micro-fluidic pump functions to bring a sample of fluid towards or away from the quantum-dot spectrometer when the spectrometer is used for analysis. In some examples the biomedical device may be an ophthalmic device. In some examples the biomedical device may be a contact lens. In some examples the biomedical device may be an electronic pill.

Implementations may include a method of analyzing analytes. The method may include fabricating a quantum-dot light emitter into a biomedical device. As well, a photodetector may be included into a biomedical device. The method may include connecting the quantum-dot emitter and photodetector to an integrated circuit controller where this controller may be capable of directing the functionality of the quantum-dot emitter and photodetector. The method may further include emitting a narrow wavelength band from the quantum-dot light emitter. The method may include receiving transmitted photons into the photodetector. In some implements the method may continue with analyzing the absorbance of an analyte based on the intensity of photons received. The biomedical device may comprise an energization element which may include a first and second current collector, a cathode, and anode and an electrolyte where the spectrometer is powered by the energization element. The method may further comprise pumping a sample of analytes into the quantum-dot spectrometer cannel before analyzing the analytes. In some implementations, the method may involve the examples where the biomedical device is a contact lens. The method may also involve examples where the biomedical device is an electronic pill.

One general aspect includes a biomedical device comprising an energization element. The biomedical device may include an external encapsulation boundary. The external encapsulation boundary may include a reentrant cavity which creates an external region that may be generally surrounded by the biomedical device while allowing fluid to flow in and out from the environment of the biomedical device. The encapsulation layers of the biomedical device may allow light to pass through them in important spectral bands. The reentrant channel may be lined by photon emitters and detectors.

In some implementations, the biomedical device may include a quantum-dot light emitter installed to emit light through one side of the sidewall of the cavity through the intervening space of the cavity. The light may further proceed through the opposite or distal side of the sidewall of the cavity. On the other side may be numerous photodetectors installed within the external encapsulation boundary. The biomedical device may also include a radio frequency transceiver and an analog-to-digital converter. A signal from the photodetector may be converted by the analog-to-digital converter into a data value that may be transmitted by the radio frequency transceiver. In some examples the biomedical device may be a contact lens or an electronic pill. In examples of an electronic pill, the pill may also comprise a release mechanism controllable to release medicament. The detector may form a feedback loop for the device and therefore may adjust the amount of medicament dispersed by the pill.

In some examples of a biomedical device comprising a QD spectrometer, the signal received at the photodetector may be converted to a digital signal and communicated to an external receiver. This external receiver may include a processor that may execute an algorithm which calculates a concentration of an analyte and then determines the concomitant release in medicament that is desired. The external receiver may transmit data and control signals to the biomedical device.

Implementations may include a biomedical device including an energization element. The biomedical device may also include an external encapsulation boundary wherein at least a portion of the boundary comprises an electrically controlled pore. The pore may be operative to allow a fluid sample to pass into the biomedical device from an external region. The biomedical device may also include a microfluidic processing chip which may mix fluid samples and reagents. Reagents in the microfluidic processing chip may include analyte specific dyes. The biomedical device may include a quantum-dot light emitter which may emit light through a portion of the microfluidic processing chip. The device may also include a photodetector installed on a distal position of the microfluidic processing chip. The device may also include a radio frequency transceiver. The implement may also include an analog to digital converter where a signal from the photodetector may be converted to a digital data value that is transmitted outside the biomedical device by the radio frequency transceiver. These examples may include biomedical devices which are contact lenses, electronic pills and electronic pills capable to release medicament based on the signal received at the photodetector.

One general aspect includes a biomedical device as an electronic pill, where the electronic pill includes a release mechanism controllable to release a quantum-dot dye into the cavity, where the dye reacts with analyte molecules and allows the quantum light emitter to excite the quantum-dot dye to emit light. The biomedical device also includes an energization element; an external encapsulation boundary, where at least a portion of the boundary includes an electrically controlled pore operative to allow a fluid sample to pass into the biomedical device from an external region; a microfluidic processing chip operative to mix the fluid sample with a reagent including an analyte specific dye; a quantum-dot light emitter installed to emit light through a portion of the microfluidic processing chip; a photodetector installed on a distal side of the microfluidic processing chip from the quantum-dot light emitter, where light emitted by the quantum-dot light emitter proceeds through a top surface of the microfluidic processing chip, through a sample analysis region of the microfluidic processing chip, through a bottom surface of the microfluidic processing chip and into the photodetector; a radio frequency transceiver; and an analog-to-digital converter, where a signal from the photodetector is converted to a digital data value that is transmitted. The electronic pill may control its release of medicament. The release of medicament may be adjusted by a controller which acts in response to receipt of converted digital data value.

In some examples the biomedical device may comprise a portion that is controllable to release a quantum-dot dye into the microfluidic processing chip. The dye may react with analyte molecules and the reaction may allow the quantum-dot light emitter to excite the quantum-dots without the presence of quenching molecules which may extinguish the characteristic emission from the quantum-dots.

### BRIEF DESCRIPTION OF THE DRAWINGS

The foregoing and other features and advantages of the invention will be apparent from the following, more particular description of preferred embodiments of the invention, as illustrated in the accompanying drawings:

FIGS. 1A-1B illustrate exemplary aspects of biocompatible energization elements in concert with the exemplary application of contact lenses.

FIG. 2 illustrates how a spectral band may be analyzed with quantum-dot based filters.

FIG. 3 illustrates a processor that may be used to implement some embodiments of the present invention.

FIG. 4 illustrates an exemplary functional structure model for a biomedical device for a Quantum-Dot Spectrometer.

FIG. 5 illustrates an exemplary Quantum-Dot Spectrometer device.

FIG. 6A illustrates a top view of an exemplary multi-piece annular shaped form insert.

FIG. 6B illustrates a first amplified partial cross sectional representation of the exemplary multi-piece annular shaped form insert of FIG. 6A.

FIG. 6C illustrates a second amplified partial cross sectional representation of the exemplary multi-piece annular shaped form insert of FIG. 6A.

FIG. 7 illustrates a magnified top view partial section of the Quantum-dot Spectrometer System of with an exemplary pumping mechanism as well as sampling regions and controlling components.

FIG. 8 illustrates a top view partial section of an exemplary Quantum-dot Spectrometer System with a fluid sample being flowed through the microfluidic analysis component.

FIG. 9 illustrates a top view section of an exemplary Quantum-dot Spectrometer System component with a waste storage element.

FIG. 10 illustrates a top view section of an exemplary pumping mechanism for a Quantum-dot Spectrometer System using lab on a chip component.

FIGS. 11 A-C illustrate an exemplary Quantum-Dot Spectrometer in a biomedical device.

FIG. 12 illustrates an exemplary quantum-dot based fluorescence dye.

FIG. 13 illustrates an exemplary flow diagram for sample analyte detection by quantum-dot based spectroscopy.

FIG. 14 illustrates exemplary method steps that may be used to monitor analyte levels of a user wearing the ophthalmic lens according to aspects of the present disclosure.

FIG. 15 illustrates exemplary method steps that may be used to treat the glucose levels of a user wearing the ophthalmic lens according to aspects of the present disclosure.

### DETAILED DESCRIPTION OF THE INVENTION

Spectroscopy utilizing quantum-dots as emission sources, filters and dyes which may be used in biomedical devices are disclosed in this application. In the following sections, detailed descriptions of various examples are described. The descriptions are exemplary embodiments only, and various modifications and alterations may be apparent to those skilled in the art. Therefore, the examples do not limit the scope of this application. Quantum-dot based spectrometers for use in biomedical devices, and the structures that contain them, may be designed for use in devices such as ophthalmic lenses and electronic pills. In some examples, spectroscopy methods utilizing quantum-dots for use in biomedical devices may be designed for use in, or proximate to, the body of a living organism.

### Glossary

In the description and claims below, various terms may be used for which the following definitions will apply:

“Anode” as used herein refers to an electrode through which electric current flows into a polarized electrical device. The direction of electric current is typically opposite to the direction of electron flow. In other words, the electrons flow from the anode into, for example, an electrical circuit.

“Binder” as used herein refers to a polymer that is capable of exhibiting elastic responses to mechanical deformations and that is chemically compatible with other energization element components. For example, binders may include electroactive materials, electrolytes, polymers, and the like.

“Biocompatible” as used herein refers to a material or device that performs with an appropriate host response in a specific application. For example, a biocompatible device does not have toxic or injurious effects on biological systems.

“Cathode” as used herein refers to an electrode through which electric current flows out of a polarized electrical device. The direction of electric current is typically opposite to the direction of electron flow. Therefore, the electrons flow into the cathode of the polarized electrical device, and out of, for example, the connected electrical circuit.

“Coating” as used herein refers to a deposit of material in thin forms. In some uses, the term will refer to a thin deposit that substantially covers the surface of a substrate it is formed upon. In other more specialized uses, the term may be used to describe small thin deposits in smaller regions of the surface.

“Electrode” as used herein may refer to an active mass in the energy source. For example, it may include one or both of the anode and cathode.

“Energized” as used herein refers to the state of being able to supply electrical current or to have electrical energy stored within.

“Energy” as used herein refers to the capacity of a physical system to do work. Many uses of the energization elements may relate to the capacity of being able to perform electrical actions.

“Energy Source” or “Energization Element” or “Energization Device” as used herein refers to any device or layer which is capable of supplying energy or placing a logical or electrical device in an energized state. The energization elements may include batteries. The batteries may be formed from alkaline type cell chemistry and may be solid-state batteries or wet cell batteries.

“Fillers” as used herein refer to one or more energization element separators that do not react with either acid or alkaline electrolytes. Generally, fillers may include substantially water insoluble materials such as carbon black; coal dust; graphite; metal oxides and hydroxides such as those of silicon, aluminum, calcium, magnesium, barium, titanium, iron, zinc, and tin; metal carbonates such as those of calcium and magnesium; minerals such as mica, montmorollonite, kaolinite, attapulgite, and talc; synthetic and natural zeolites such as Portland cement; precipitated metal silicates such as calcium silicate; hollow or solid polymer or glass microspheres, flakes and fibers; and the like.

“Functionalized” as used herein refers to making a layer or device able to perform a function including, for example, energization, activation, and/or control.

“Ionizing Salt” as used herein refers to an ionic solid that will dissolve in a solvent to produce dissolved ions in solution. In numerous examples, the solvent may comprise water.

“Mold” as used herein refers to a rigid or semi-rigid object that may be used to form three-dimensional objects from uncured formulations. Some exemplary molds include two mold parts that, when opposed to one another, define the structure of a three-dimensional object.

“Power” as used herein refers to work done or energy transferred per unit of time.

“Rechargeable” or “Re-energizable” as used herein refer to a capability of being restored to a state with higher capacity to do work. Many uses may relate to the capability of being restored with the ability to flow electrical current at a certain rate for certain, reestablished time periods.

“Reenergize” or “Recharge” as used herein refer to restoring to a state with higher capacity to do work. Many uses may relate to restoring a device to the capability to flow electrical current at a certain rate for a certain reestablished time period.

“Released” as used herein and sometimes referred to as “released from a mold” means that a three-dimensional object is either completely separated from the mold, or is only loosely attached to the mold, so that it may be removed with mild agitation.

“Stacked” as used herein means to place at least two component layers in proximity to each other such that at least a portion of one surface of one of the layers contacts a first surface of a second layer. In some examples, a coating, whether for adhesion or other functions, may reside between the two layers that are in contact with each other through said coating.

“Traces” as used herein refer to energization element components capable of connecting together the circuit components. For example, circuit traces may include copper or gold when the substrate is a printed circuit board and may typically be copper, gold or printed film in a flexible circuit. A special type of “Trace” is the current collector. Current collectors are traces with electrochemical compatibility that make the current collectors suitable for use in conducting electrons to and from an anode or cathode in the presence of electrolyte.

The methods and apparatus presented herein relate to forming biocompatible energization elements for inclusion within or on flat or three-dimensional biocompatible devices. A particular class of energization elements may be batteries that are fabricated in layers. The layers may also be classified as laminate layers. A battery formed in this manner may be classified as a laminar battery.

There may be other examples of how to assemble and configure batteries according to the present invention, and some may be described in following sections. However, for many of these examples, there are selected parameters and characteristics of the batteries that may be described in their own right. In the following sections, some characteristics and parameters will be focused upon.

Recent developments in biomedical devices including, for example, ophthalmic lenses, have enabled functionalized biomedical devices that may be energized. The energized biomedical devices may comprise the necessary elements to collect and analyze analytes of users using embedded micro-electronics. Additional functionality using micro-electronics may include, for example, audio, visual, and haptic feedback to the user. In some embodiments, the quantum-dot spectrometers for use in biomedical devices may be in wireless communication with one or more wireless device(s) and receive signal data that may be used in real time for the determination of an abnormal analyte concentration and correlated cause. The wireless device(s) may include, for example, a smart phone device, a tablet, a personal computer, a FOB, an MP3 player, a PDA, and other similar devices.

### Energized Ophthalmic Device

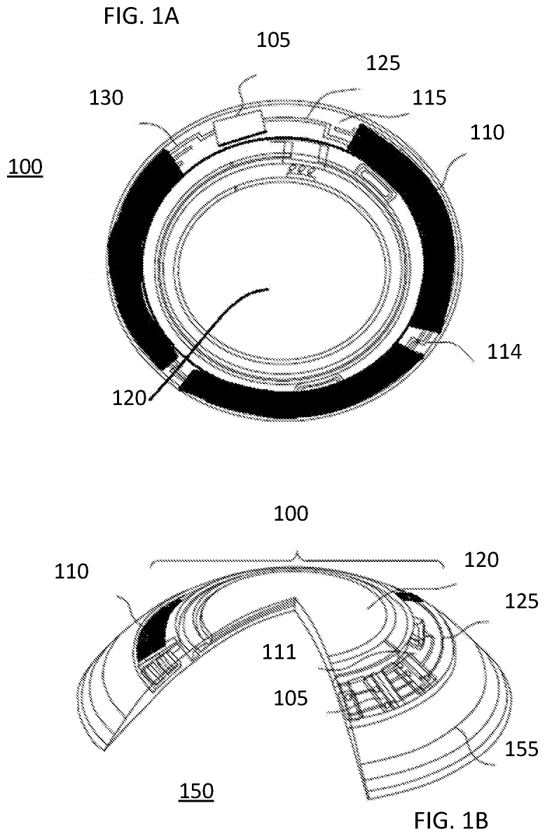

Referring to FIG. 1A, an exemplary embodiment of a media insert 100 for an energized ophthalmic device and a corresponding energized ophthalmic device 150 (FIG. 1B) are illustrated to provide an example of an energized biomedical device structure that may support the operation of quantum-dot based spectroscopy. The media insert 100 may comprise an optical zone 120 that may or may not be functional to provide vision correction. Where the energized function of the ophthalmic device is unrelated to vision, the optical zone 120 of the media insert may be void of material. In some exemplary embodiments, the media insert may include a portion not in the optical zone 120 comprising a substrate 115 incorporated with energization elements 110 (power source) and electronic components 105, such as a spectrometer. The energization elements 110 may be connected to a circuit element that may have its own substrate 111 upon which interconnect features 125 may be located. The circuit, which may be in the form of an integrated circuit, may be electrically and physically connected to the substrate 111 and its interconnect features 125. The energization elements 110 may have their own interconnect features to join together elements as may be depicted underlying the region of interconnect 114.

In some exemplary embodiments, a power source or energization elements 110 (for example a battery) and a load such as electronic components 105 (for example a spectrometer) may be attached to the substrate 115. Conductive traces called interconnect features 125 and 130 may electrically interconnect the electronic components 105 and the energization elements 110. The media insert 100 may be fully encapsulated to protect and contain the energization elements 110, interconnect features 125, and electronic components 105, such as a spectrometer. In some exemplary embodiments, the encapsulating material may be semi-permeable, for example, to prevent specific substances, such as water, from entering the media insert and to allow specific substances, such as ambient gasses or the byproducts of reactions within energization elements, to penetrate or escape from the media insert.

In some exemplary embodiments, as depicted in FIG. 1B, the media insert 100 may be included in an ophthalmic device 150, which may comprise a polymeric biocompatible material. The ophthalmic device 150 may include a rigid center, soft skirt design wherein the central rigid optical element comprises the media insert 100. In some specific embodiments, the media insert 100 may be in direct contact with the atmosphere and the corneal surface on respective anterior and posterior surfaces, or alternatively, the media insert 100 may be encapsulated in the ophthalmic device 150. The periphery 155 of the ophthalmic device 150 or lens may be a soft skirt material, including, for example, a hydrogel material. The infrastructure of the media insert 100 and the ophthalmic device 150 may provide an environment for numerous embodiments involving fluid sample processing with quantum-dot based analysis elements while isolating the internal components from the biomedical environment surrounding the insert.

### Fluorescence Based Probe Elements for Analyte Analysis

Various types of analytes may be detected and analyzed using fluorescence based analysis techniques. A subset of these techniques may involve the direct fluorescence emission from the analyte itself. A more generic set of techniques relate to fluorescence probes that have constituents that bind to analyte molecules, and in so binding, alter a fluorescence signature. For example, in Förster Resonance Energy Transfer (FRET), probes are configured with a combination of two fluorophores that may be chemically attached to interacting proteins. The distance of the fluorophores from each other can affect the efficiency of a fluorescence signal emanating therefrom.

One of the fluorophores may absorb an excitation irradiation signal and can resonantly transfer the excitation to electronic states in the other fluorophore. The binding of analytes to the attached interacting proteins may disturb the geometry and cause a change in the fluorescent emission from the pair of fluorophores. Binding sites may be genetically programmed into the interacting proteins, and for example, a binding site, which is sensitive to glucose, may be programmed. In some cases, the resulting site may be less sensitive or non-sensitive to other constituents in interstitial fluids of a desired sample.

The binding of an analyte to the FRET probes may yield a fluorescence signal that is sensitive to glucose concentrations. In some exemplary embodiments, the FRET based probes may be sensitive to as little as a 10 uM concentration of glucose and may be sensitive to concentrations of up to hundreds of micromolar. Various FRET probes may be genetically designed and formed. The resulting probes may be configured into structures that may assist analysis of interstitial fluids of a user. In some exemplary embodiments, the probes may be placed within a matrix of material that is permeable to the interstitial fluids and their components, for example, the FRET probes may be assembled into hydrogel structures. In some exemplary embodiments, these hydrogel probes may be included into the hydrogel based processing of ophthalmic contact lenses in such a manner that they may reside in a hydrogel encapsulation that is immersed in tear fluid when worn upon the eye. In other exemplary embodiments, the probe may be inserted in the ocular tissues just above the sclera. A hydrogel matrix comprising fluorescence emitting analyte sensitive probes may be placed in various locations that are in contact with bodily fluids containing an analyte.

In the examples provided, the fluorescence probes may be in contact with interstitial fluid of the ocular region near the sclera. In these cases, where the probes are invasively embedded, a sensing device may provide a radiation signal incident upon the fluorescence probe from a location external to the eye such as from an ophthalmic lens or a hand held device held in proximity to the eye.

In other exemplary embodiments, the probe may be embedded within an ophthalmic lens in proximity to a fluorescence-sensing device that is also embedded within the ophthalmic lens. In some exemplary embodiments, a hydrogel skirt may encapsulate both an ophthalmic insert with a fluorescence detector as well as a FRET based analyte probe.

### Quantum-Dot Spectroscopy

Small spectroscopy devices may be of significant aid in creating biomedical devices with the capability of measuring and controlling concentrations of various analytes for a user. For example, the metrology of glucose may be used to control variations of the material in patients and after treatments with medicines of various kinds. Current microspectrometer designs mostly use interference filters and interferometric optics to measure spectral responses of mixtures that contain materials that absorb light. In some examples a spectrometer may be formed by creating an array composed of quantum-dots. A spectrometer based on quantum-dot arrays may measure a light spectrum based on the wavelength multiplexing principle. The wavelength multiplexing principle may be accomplished when multiple spectral bands are encoded and detected simultaneously with one filter element and one detector element, respectively. The array format may allow the process to be efficiently repeated many times using different filters with different encoding so that sufficient information is obtained to enable computational reconstruction of the target spectrum. An example may be illustrated by considering an array of light detectors such as that found in a CCD camera. The array of light sensitive devices may be useful to quantify the amount of light reaching each particular detector element in the CCD array. In a broadband spectrometer, a plurality, sometimes hundreds, of quantum-dot based filter elements are deployed such that each filter allows light to pass from certain spectral regions to one or a few CCD elements. An array of hundreds of such filters laid out such that an illumination light passed through a sample may proceed through the array of QD Filters and on to a respective set of CCD elements for the QD filters. The simultaneous collection of spectrally encoded data may allow for a rapid analysis of a sample.

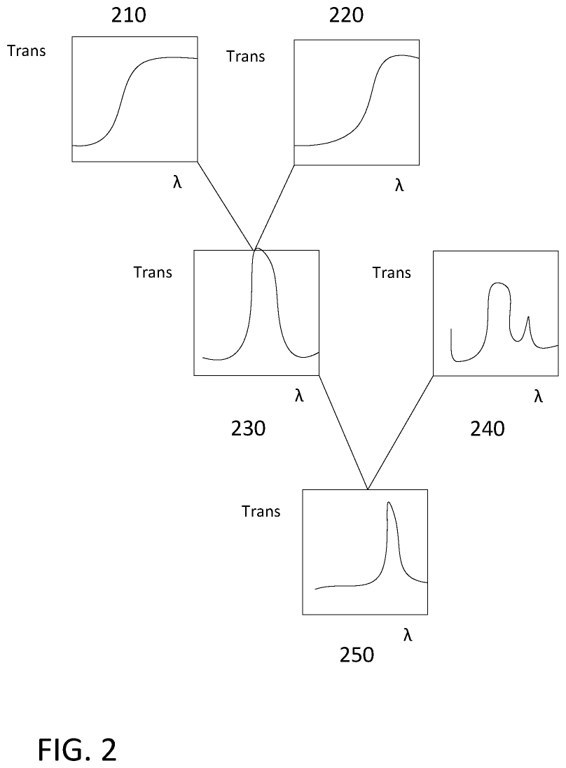

Narrow band spectral analysis examples may be formed by using a smaller number of QD filters surrounding a narrow band. In FIG. 2 an illustration of how a spectral band may be observed by a combination of two filters is illustrated. It may also be clear that the array of hundreds of filters may be envisioned as a similar concept to that in FIG. 2 repeated may times.

If FIG. 2, a first QD filter 210 may have an associated spectral transmission response as illustrated and indicated as Trans. A second QD filter 220 may have a shifted associated spectral transmission associated with a different nature of the quantum-dots included in the filter, for example the QDs may have a larger diameter in the QD filter of 220. The difference curve of a flat irradiance of light of all wavelength (white light) may result from the difference of the absorption result from light that traverses filter 220 and that traverses filter 210. Thus, the effect of irradiating through these two filters is that the difference curve would indicate spectral response in the band 230 depicted. When an analyte is introduced into the light path of the spectrometer, where the analyte has an absorption band in the UV/Visible spectrum, and possible in the infrared, the result would be to modify the transmission of light in that spectral band as shown by spectrum 240. The difference from 230 to 240 results in a transmission spectrum 250 for the analyte in the region defined by the two quantum-dot filters. Therefore, a narrow spectral response may be obtained by a small number of filters. In some examples, redundant coverage by different filter types of the same spectral region may be employed to improve the signal to noise characteristics of the spectral result.

The absorption filters based on QDs may include QDs that have quenching molecules on their surfaces. These molecules may stop the QD from emitting light after it absorbs energy in appropriate frequency ranges. More generally, the QD filters may be formed from nanocrystals with radii smaller than the bulk exciton Bohr radius, which leads to quantum confinement of electronic charges. The size of the crystal is related to the constrained energy states of the nanocrystal and generally decreasing the crystal size has the effect of a stronger confinement. This stronger confinement affects the electronic states in the quantum-dot and results in an increased the effective bandgap, which results in shifting to the blue wavelengths both of both optical absorption and fluorescent emission. There have been many spectral limited sources defined for a wide array of quantum-dots that may be available for purchase or fabrication and may be incorporated into biomedical devices to act as filters. By deploying slightly modified QDs such as by changing the QD's size, shape and composition it may be possible to tune absorption spectra continuously and finely over wavelengths ranging from deep ultraviolet to mid-infrared. QDs can also be printed into very fine patterns.

### Diagrams for Electrical and Computing System

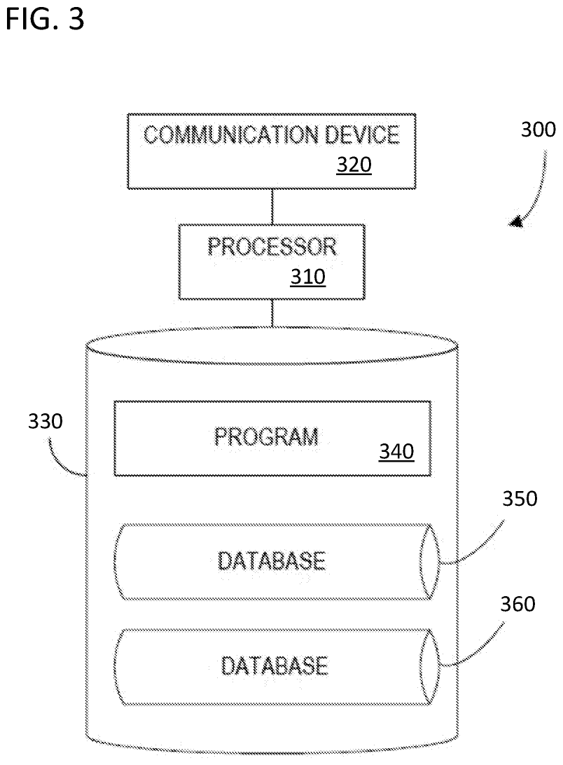

Referring now to FIG. 3, a schematic diagram of a processor that may be used to implement some aspects of the present disclosure is illustrated. The controller 300 may include one or more processors 310, which may include one or more processor components coupled to a communication device 320. In some embodiments, a controller 300 may be used to transmit energy to the energy source placed in the device.

The processors 310 may be coupled to a communication device 320 configured to communicate energy via a communication channel. The communication device 320 may be used to electronically communicate with components within the media insert, for example. The communication device 320 may also be used to communicate, for example, with one or more controller apparatus or programming/interface device components.

The processor 310 is also in communication with a storage device 330. The storage device 330 may comprise any appropriate information storage device, including combinations of magnetic storage devices, optical storage devices, and/or semiconductor memory devices such as Random Access Memory (RAM) devices and Read Only Memory (ROM) devices.

The storage device 330 may store a program 340 for controlling the processor 310. The processor 310 performs instructions of a software program 340, and thereby operates in accordance with the present invention. For example, the processor 310 may receive information descriptive of media insert placement, active target zones of the device. The storage device 330 may also store other pre-determined biometric related data in one or more databases 350 and 360. The database may include, for example, predetermined retinal zones exhibiting changes according to cardiac rhythm or an abnormal condition correlated with the retinal vascularization, standard measurement thresholds, metrology data, and specific control sequences for the system, flow of energy to and from a media insert, communication protocols, and the like. The database may also include parameters and controlling algorithms for the control of the biometric based monitoring system that may reside in the device as well as data and/or feedback that can result from their action. In some embodiments, that data may be ultimately communicated to/from an external reception wireless device.

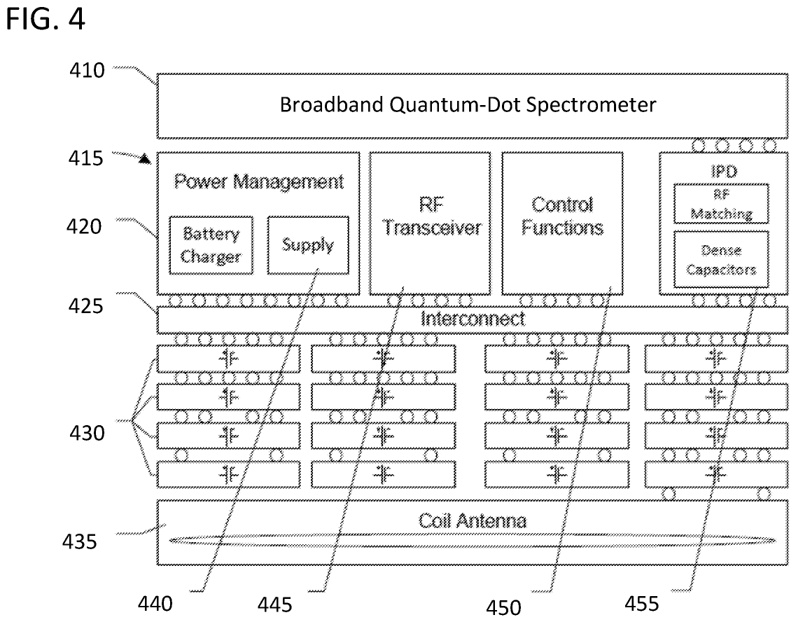

In some embodiments according to aspects of the disclosure, a single and/or multiple discrete electronic devices may be included as discrete chips. In other embodiments, energized electronic elements may be included in a media insert in the form of stacked integrated components. Referring now to FIG. 4, a schematic diagram of an exemplary cross section of a stacked die integrated components implementing a quantum-dot spectrometer system 410 is depicted. The quantum-dot spectrometer may be, for example, a glucose monitor, a retinal vascularization monitor, a visual scanning monitor, or any other type of system useful for providing spectrophometric information about the user. In particular, a media insert may include numerous layers of different types which are encapsulated into contours consistent with the environment that they will occupy. In some embodiments, these media inserts with stacked integrated component layers may assume the entire shape of the media insert. Alternatively in some cases, the media insert may occupy just a portion of the volume within the entire shape.

As shown in FIG. 4, there may be thin film batteries 430 used to provide energization. In some embodiments, these thin film batteries 430 may comprise one or more of the layers that may be stacked upon each other with multiple components in the layers and interconnections there between. The batteries 430 are depicted as thin film batteries for exemplary purposes, there may be numerous other energization elements consistent with the embodiments herein including operation in both stacked and non-stacked embodiments. As a non-limiting alternative example cavity based laminate form batteries with multiple cavities may perform equivalently or similarly to the depicted thin film batteries.

In some embodiments, there may be additional interconnections between two layers that are stacked upon each other. In the state of the art there may be numerous manners to make these interconnections; however, as demonstrated the interconnection may be made through solder ball interconnections between the layers. In some embodiments only these connections may be required; however, in other cases the solder balls may contact other interconnection elements, as for example with a component having through layer vias.

In other layers of the stacked integrated component media insert, a layer 425 may be dedicated for the interconnections of two or more of the various components in the interconnect layers. The interconnect layer 425 may contain, vias and routing lines that can pass signals from various components to others. For example, interconnect layer 425 may provide the various battery elements connections to a power management unit 420 that may be present in a technology layer 415. The power management unit 420 may have circuitry dedicated to supplying voltage sources with controlled characteristics 440. Other components in the technology layer 415 may include, for example, a transceiver 445, control components 450 and the like. In addition, the interconnect layer 425 may function to make connections between components in the technology layer 415 as well as components outside the technology layer 415; as may exist for example in the integrated passive device 455. There may be numerous manners for routing of electrical signals that may be supported by the presence of dedicated interconnect layers such as interconnect layer 425.

In some embodiments, the technology layer 415, like other layer components, may be included as multiple layers as these features represent a diversity of technology options that may be included in media inserts. In some embodiments, one of the layers may include CMOS, BiCMOS, Bipolar, or memory based technologies whereas the other layer may include a different technology. Alternatively, the two layers may represent different technology families within a same overall family; as for example one layer may include electronic elements produced using a 0.5 micron CMOS technology and another layer may include elements produced using a 20 nanometer CMOS technology. It may be apparent that many other combinations of various electronic technology types would be consistent within the art described herein.

In some embodiments, the media insert may include locations for electrical interconnections to components outside the insert. In other examples, however, the media insert may also include an interconnection to external components in a wireless manner. In such cases, the use of antennas in an antenna layer 435 may provide exemplary manners of wireless communication. In many cases, such an antenna layer 435 may be located, for example, on the top or bottom of the stacked integrated component device within the media insert.

In some of the embodiments discussed herein, the energization elements such as batteries 430 may be included as elements in at least one of the stacked layers themselves. It may be noted as well that other embodiments may be possible where the battery elements 430 are located externally to the stacked integrated component layers. Still further diversity in embodiments may derive from the fact that a separate battery or other energization component may also exist within the media insert, or alternatively these separate energization components may also be located externally to the media insert. In these examples, the functionality may be depicted for inclusion of stacked integrated components, it may be clear that the functional elements may also be incorporated into biomedical devices in such a manner that does not involve stacked components and still be able to perform functions related to the embodiments herein.

Components of the quantum-dot spectrometer system 410 may also be included in a stacked integrated component architecture. In some embodiments, the quantum-dot spectrometer system 410 components may be attached as a portion of a layer. In other embodiments, the entire quantum-dot spectrometer system 410 may also comprise a similarly shaped component as the other stacked integrated components. In some alternative examples, the components may not be stacked but laid out in the peripheral regions of the ophthalmic device or other biomedical device, where the general functional interplay of the components may function equivalently however the routing of signals and power through the entire circuit may differ.

When constructing a quantum-dot spectrometer system 410 in a biomedical device, size may be an integral factor. Quantum-dot emitters may be fashioned in a manner similar to the formation of light emitting diodes. Layers of materials may surround the quantum-dots to create light emitting diodes with the quantum-dots. Organic layers may act as electron donors and as hole donors into the quantum-dot layer. In a non-limiting example, the QDs may be sandwiched between electron transport layers and hole transport layers. Application of electric potential to electrodes connected to the electron transport layer and the hold transport layer excite the QD into photoluminescence at a wavelength band characteristic of the QDs. Examples of electron transport layers and hole transport layers may include tris-(8-hydroxyquinoline) aluminum; bathocuproine; 4,4′-N,N′-dicarbazolylbiphenyl; poly(2-(6-cyano-6′-methylheptyloxy)-1,4-phenylene); poly[(9,9-dihexylfluorenyl-2,7-diyl)-co-(1,4-{benzo-[2,1′,3]thiadiazole})]; poly[2-methoxy-5-(2-ethylhexyloxy)-1,4-phenylenevinylene]; 4,4-bis[N-(1-naphyl)-N-phenylamino]biphenyl; 2-(4-biphenylyl)-5-(4-tertbutylphenyl)-1,3,4 oxadiazole; poly-3,4-ethylene dioxythiophene; poly(9,9′-dioctylfluorene-co-N-(4-butylphenyl)diphenylamine; perfluoro-cyclobutane; poly(phenylene vinylene); 3-(4-Biphenylyl)-4-phenyl-5-tertbutylphenyl-1, 2, 4-triazole; Poly[(9,9-dioctylfluorenyl-2,70diyl)-co-(4-4′-(N-(4-secbutylphenyl)) diphenylamine)]; 1,3,5-tris(N-phenylbenzimidazole-2-yl)-benzene; and N, N′-diphenyl-N, N′-bis(3-methylphenyl)-(1, 1′-biphenyl)-4, 4′-diamine as non-limiting examples.

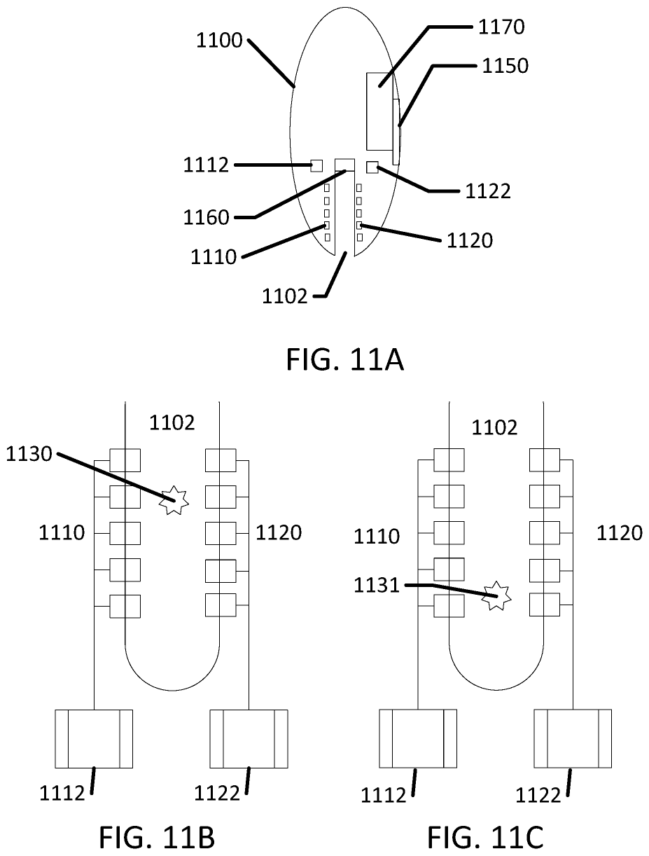

Once excited by a current (such as from the power management unit 420), the quantum-dot layer may emit light at a designed specified wavelength from the quantum-dot spectrometer system 410. The emitted light may interact with an outside environment, or with a specific sample or samples in the environment, wherein the sample or samples may absorb the emitted light at certain wavelengths. The quantum-dot spectrometer may then receive the remaining light, which has been transmitted through the sample or samples, in a quantum-dot detector (refer to FIG. 11A for one exemplary embodiment) within the quantum-dot spectrometer system 410.

Similarly, millimeter or nanometer sized quantum-dot detectors may be implemented into a quantum-dot spectrometer system 410 (see also, FIG. 11A, at 1120). Current quantum-dot detectors may rely on charged-coupled devices (CCD); however, CCDs do not currently provide the size scale required for millimeter- or nanometer sized quantum-dot spectrometers. Rather, smaller photodiode arrays may be used to achieve the size requirements. Photodiodes are semiconductor devices that convert light into energy. Millimeter or nanometer sized photodiodes may be constructed through photolithographic means.

### Biomarkers/Analytical Chemistry

A biomarker, or biological marker, generally refers to a measurable indicator of some biological state or condition. The term is also occasionally used to refer to a substance the presence of which indicates the existence of a living organism. Further, life forms are known to shed unique chemicals, including DNA, into the environment as evidence of their presence in a particular location. Biomarkers are often measured and evaluated to examine normal biological processes pathogenic processes, or pharmacologic responses to a therapeutic intervention. In their totality, these biomarkers may reveal vast amounts of information important to the prevention and treatment of disease and the maintenance of health and wellness.

Biomedical devices configured to analyze biomarkers may be utilized to quickly and accurately reveal one's normal body functioning and assess whether that person is maintaining a healthy lifestyle or whether a change may be required to avoid illness or disease. Biomedical devices may be configured to read and analyze proteins, bacteria, viruses, changes in temperature, changes in pH, metabolites, electrolytes, and other such analytes used in diagnostic medicine and analytical chemistry.

Ophthalmic Insert Devices and Other Biomedical Devices with Quantum-Dot Spectrometer

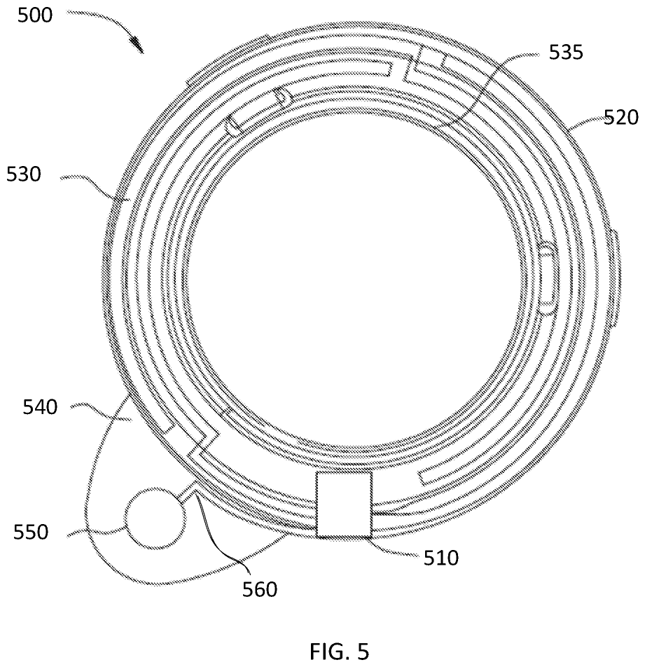

Referring to FIG. 5, an ophthalmic insert 500 is demonstrated including components that may form an exemplary quantum-dot (QD) spectrometer system. The demonstrated ophthalmic insert is shown in an exemplary annular form having an internal border of 535 and an external border of 520. In addition to energization elements such as the battery power source 530, control circuitry 510, and interconnect features 560 there may be a broadband QD spectrometer system 550, which in certain exemplary embodiments may be positioned on a flap 540. The flap 540 may be connected to the insert 500 or be an integral, monolithic extension thereof. The flap 540 may properly position the broadband QD spectrometer system 550 when an ophthalmic device comprising a QD detector is worn. The flap 540 may allow the broadband QD spectrometer system 550 to overlap with portions of the user's eye away from the optic zone. The broadband QD spectrometer system 550 may be capable of determining an analyte, in terms of its presence or its concentration, in a fluid sample.

For a broadband QD spectrometer, an analyte sample may be exposed to an excitation light source which may be passed through an array of different QD filters. This light source with the array of filters may be located within the body of the analytical system. In some exemplary embodiments, the light source may comprise a solid-state device or devices similar to a light emitting diode (LED). The QD filtered light source may be irradiated through a sample and may reflect back on some tissue layers to a detector array

An electronic control bus of interconnect features 560 may provide the signals to the light source or sources and return signals from the detectors. The powered electronic component may provide the signals and power aspects. The exemplary embodiment of FIG. 5, illustrates a battery power source 530 to the electronic circuitry 510. In other exemplary embodiments, energization may also be provided to the electronic circuitry by the coupling of energy through wireless manners such as radiofrequency transfer or photoelectric transfer.

### Methods of Detecting—Microfluidic System

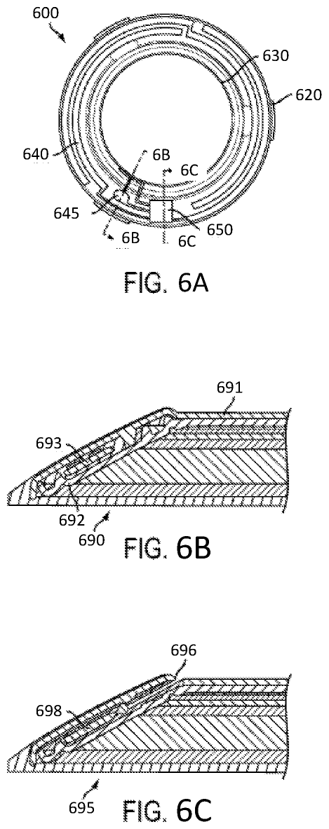

A Microfluidic System may be utilized to pull sample from an outside environment into the biomedical device in order to analyze the sample in a more controlled fashion within an internal region defined by the insert pieces. Referring now to FIG. 6A, a top view of an exemplary multi-piece annular shaped insert 600 is depicted. As depicted, the exemplary multi-piece annular shaped insert 600 may be a ring of material around a central optical zone that is devoid of material. Moreover, the annular shaped insert 600 may be defined by an exterior extent 620 and an internal annulus edge 630. Included in between the exterior extend 620 and the internal annulus edge 630 may be found energization elements 640, interconnect features 645 of various types and/or an electronic circuit element 650.

Referring now to FIG. 6B, a first amplified partial cross sectional representation 690 of the exemplary multipiece annular shaped form insert 600 of FIG. 6A is depicted. The cross section 690 reveals that the annular shaped insert 600 as a combination of a front insert piece 691 and a rear insert piece 692. As depicted, in some embodiments, the front insert piece 691 and the rear insert piece 692 may be joined and sealed together. In different embodiments, other structural features and means can be implemented to join both pieces together. Also shown in an encapsulated location may be an integrated circuit element 693 connected to interconnection elements.

Referring now to FIG. 6C, a second amplified partial cross sectional representation 695 of the exemplary multipiece annular shaped form insert 600 of FIG. 6A is depicted. In particular, in other sections/embodiments, a different type of structure may be found, as depicted in cross section 695. As shown, it may be observed so that there may be a gap or pore 696 that is formed to allow some portion of the interior of the annular shaped insert 600 to be open to an external environment. There may be numerous components 698 that may connect to this opening, and can themselves be encapsulated within the annular shaped insert 600. Accordingly, this ability to allow component(s) 698 situated within the annular shaped insert 600 to controllably interface with fluids and/or gasses in their exterior environment can, in some embodiments, enable for the incorporation of QD spectrometer elements within ophthalmic device.

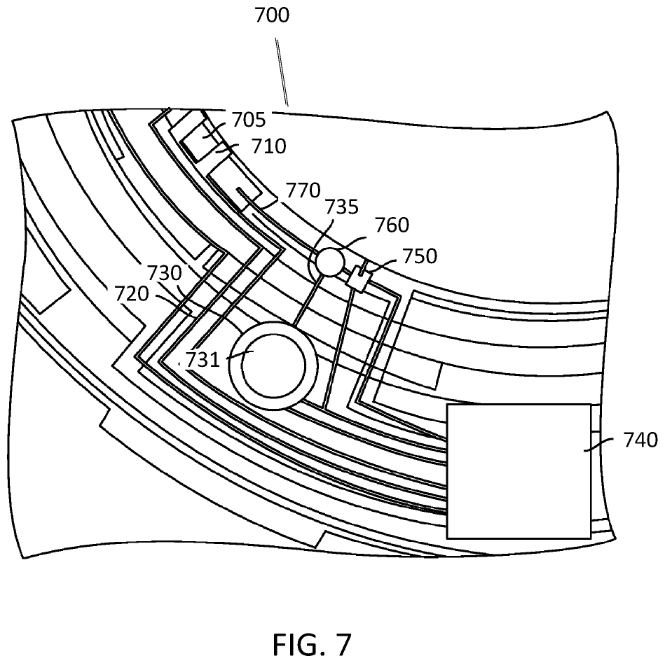

Referring now to FIG. 7, a top view of a section of a Microfluidic Analytical System 700 is illustrated with an exemplary pumping mechanism 760 as well as sampling regions and controlling components is depicted. As shown, in some embodiments control circuitry 740 may be electrically connected to components of the micro fluidic analytical system through interconnect(s) 720. A control element 750 for a pore (not shown) may be included and be useful for connecting the Microfluidic Analytical System 700 to fluid (not shown) outside of the insert. Exemplary aspects of different designs of pores may be found in following sections; however, the pore may allow fluid samples to be passed from outside the insert environment to a pumping element 760.

In some embodiments, the pumping element 760 may have an activating or driving component 730 that may be capable of engaging the pump element 760. In one example, the pump element 760 may comprise a flexible and collapsible membrane that may be activated by the application of pressure upon the membrane. There may be numerous manners for driving the application of pressure upon the membrane. For example, a fluid may fill a cavity 731 and flow through a tube 735 connecting the cavity 731 to the pumping element 760. Accordingly, the cavity 731 may include features allowing the application of pressure upon the fluid contained within. For example, piezoelectric components may be used to expand volume on the application of voltage thus pressurizing the contained fluid. In other embodiments, thermo-compressive materials may respond to a temperature change that may be controlled by the application of electric energy to a heating element. In a yet another embodiment, an Electrowetting on Dielectric (EWOD) component may exert a pressure on the fluid by a change in the wetting characteristics of a surface in cavity 731 upon the application of a potential. There may also be other means of driving a pump mechanism that may also be directly engaged at the pump element 760 itself. Still further diversity may derive from the use of EWOD components to influence the flow of fluids themselves rather than the use of mechanical pumping means.

The pump element 760 may force fluid to flow through a channel 770 and subsequently into an analyzing chamber 705 of the Microfluidic Analytical System 700. Further detail of the components in such chambers 705 will be described in following sections, but briefly stated the fluid may flow through the analyzing chamber 705 and cause influences to occur on electrode(s) 710 which may be part of the components.

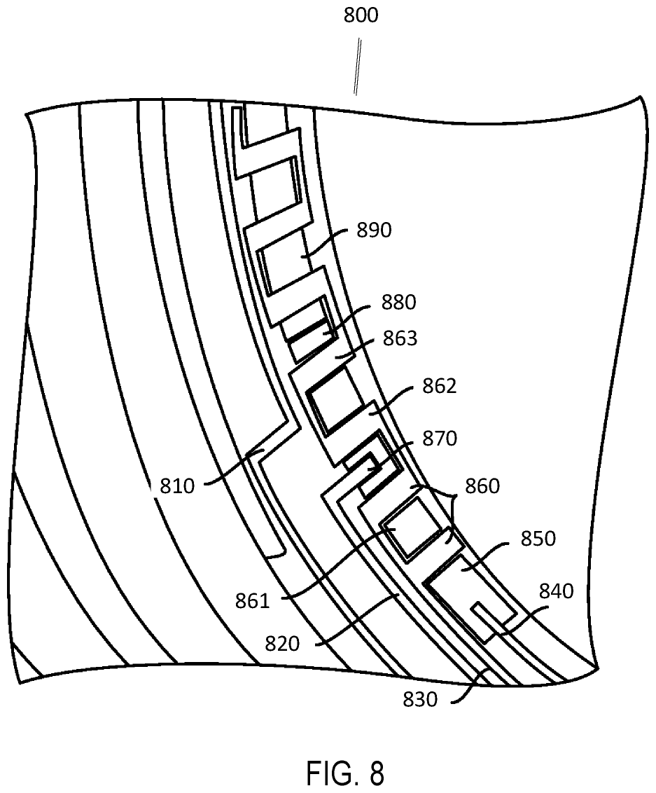

Referring now to FIG. 8, a top view partial section of an exemplary Microfluidic Analytical System 800 with a fluid sample being flowed through the microfluidic analysis component is illustrated. Because of the nature of an annular system, the components may be observed to be deployed in a curvilinear fashion as there may be numerous details that change in a curvilinear system including, for example, the exact shapes of electrodes and chamber cross sections. In other embodiments, however, linear analytical systems may be formed that have dimensions that allow them to fit in the ocular environment. Further, in additional embodiments, regardless of the nature of the system along the analysis chamber, the entire substrate that the chamber rests upon can be curved allowing it to rest upon the roughly spherical surface of an eye. The details of the three dimensional nature of the analysis chamber may factor into models related to the performance of the systems. For illustration purposes, however, this description declares these nuances, but will illustrate an exemplary embodiment by curving the features of a linear Micro fluidic Analytical System 800. Depicted in the portion of the Micro fluidic Analytical System 800, a micro-channel 850 for receiving and transporting fluid samples is shown. These fluid samples may be pumped, for example, by the previously discussed pumping system (e.g. 760 in FIG. 7) from an external location. For example, fluid samples may be sampled from ocular fluid that may surround a contact lens containing the Microfluidic Analytic System 800. An analyte sensor 870 may be found for example along the micro-channel. This analyte sensor 870 may be capable of performing one or more of: an electrochemical analysis step, a photometric analysis step or other analytical steps upon fluid samples. In some examples QD spectrometry may be performed in these regions. In an exemplary embodiment, the analysis step may relate to a photometric sensing of glucose concentration based on a fluorescence sensor typology using one or more components. In another example, the sensor may detect the presence of reaction products from a glucose oxidase interaction with portions of the analyte sensor 870 and the fluid sample. There may be numerous electrical interconnections 820 which connect the sensing element 870 to control electronics.

Fluid may flow into the micro-channel 850 from a pump channel 840. As the fluid flows into the micro-channel 850 it may displace other fluid in a particular region, or on an initial use may displace ambient gas in the channel. As a fluid flows, it may be sensed by a pre-sensor micro-channel portion comprising electrodes 860 and 861 as well as a post-sensor portion comprising electrodes 862 and 863, In some embodiments the measurement of impedance between electrodes such as 860 and 861 may be used to sense the flow of material. In other embodiments, the resistance of a chain of electrodes 862 and 863 may be altered by the presence of a fluid within the micro-channel 850, or the presence of a front between two fluids of different characteristics residing in the micro-channel 850. A fluid 880 may flow through the micro-channel from an empty region of the micro-channel 890 to be sampled. Alternatively, micro-channel portion at 890 may represent a different solution of fluid that may for example have different concentration of electrolytes, and therefore, conductivity than that of typical tear fluid.

In general, measuring impedances, or ohmic resistances, between position electrodes 860-863 in embodiments of the present invention may be accomplished by applying a voltage therebetween and measuring the resulting current. Either a constant voltage or an alternating voltage may be applied between the position electrodes 860-863 and the resulting direct current (DC) or alternating current (AC), respectively, measured. The resulting DC or AC current may then be used to calculate the impedance or ohmic resistance. Furthermore, one skilled in the art will recognize that measuring impedance may involve measuring both an ohmic drop (i.e., resistance [R] in Ohms or voltage/current) and measuring capacitance (i.e., capacitance in Farads or coulombs/volt). In practice, impedance may be measured, for example, by applying an alternating current to the position electrode(s) 860-863 and measuring the resulting current. At different frequencies of alternating current, either resistive or capacitive effects prevail in determining the measured impedance. The pure resistive component can prevail at lower frequencies while the pure capacitive component can prevail at higher frequencies. To distinguish between the resistive and capacitive components, the phase difference between the applied alternating current and the measured resulting current can be determined. If there is zero phase shift, the pure resistive component is prevailing. If the phase shift indicates that the current lags the voltage, then the capacitive component is significant. Therefore, depending on the frequency of an applied alternating current and position electrode configuration, it can be beneficial to measure either resistance or a combination of resistance and capacitance.

Referring back to the specific example of FIG. 8, impedance measurements may be performed by, for example, applying an alternating voltage between first position electrode 830 and a final position electrode connection 810 and measuring the resulting alternating current. Since the chain of electrodes including 860, 861, 862 and 863 can be a portion of a capacitor, (along with any substance [e.g., air or a liquid sample] within micro-channel 850 between subsequent position electrodes and any layers that may be separating the position electrodes from direct contact with the fluid in the micro-channel 850), the measured current may be used to calculate the impedance. The presence or absence of a liquid sample in micro-channel 850, 890 between electrodes will affect the measured current and impedance. The frequency and amplitude of the alternating voltage applied between a first and second position electrodes 860-863 can be predetermined such that the presence of a liquid sample between a first and second position electrodes 860-863 may be detected by a significant increase in measured current.

With respect to the measurement of impedance or resistance, the magnitude of the applied voltage can be, for example, in range from about 10 m V to about 2 volts for the circumstance of an ophthalmic tear fluid sample and carbon based or silver-based ink position electrodes. The lower and upper limits of the applied voltage range are dependent on the onset of electrolysis or electrochemical decomposition of the liquid sample. In instances where an alternating voltage is employed, the alternating voltage can be applied, for example, at a frequency that results in a negligible net change in the liquid sample's properties due to one or more electrochemical reaction. Such a frequency range can be, for example, from about 10 Hz to about 100 kHz with a voltage waveform symmetrical around 0 Volts (i.e., the RMS value of the alternating voltage is approximately zero).

As depicted, analyte sensor 870 and position electrodes 860-863 can each be in operative communication with the micro-channel 850. It should be noted that position electrodes 860-863 employed in embodiments of the present invention can be formed of any suitable conductive material known to those skilled in the art, including conductive materials conventionally used as analytical electrode materials and, in particular, conductive materials known as suitable for use in flexible circuits, photolithographic manufacturing techniques, screen printing techniques and flexo-printing techniques. Suitable conductive materials include, for example, carbon, noble metals (e.g., gold, platinum and palladium), noble metal alloys, conductive potential-forming metal oxides and metal salts. Position electrodes can be formed, for example, from conductive silver ink, such as the commercially available conductive silver ink Electrodag 418 SS.

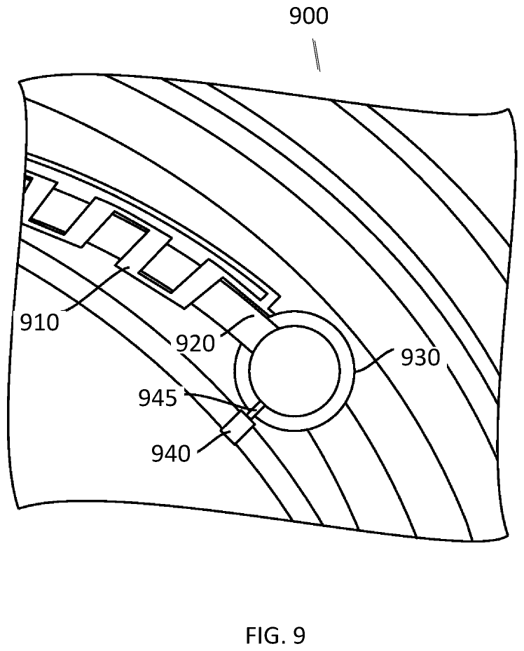

Referring now to FIG. 9, a top view section of an exemplary Microfluidic Analytical System component 900 with a waste or fluid retention vessel 930 is depicted. In the exemplary embodiments, electrode 910 for measuring the flow rate of fluid in the system may be an end electrode of many others (not depicted in FIG. 9). Fluid may flow through the microchannel 920 and continue to a fluid retention vessel 930. The fluid retention vessel may be used, for example, for higher volume of fluid analysis therein. In some embodiments, a pore 940 may include a pore control element 945 for connecting the fluid retention vessel 930, which may be also be used as a waste storage element, 930 to regions located external to the insert. In addition, in some embodiments the pore control element 945 connection may be useful for equalizing gas pressure as the microfluidic components fill with fluid. In other embodiments, the pore 940 and pore control element 945 may be useful for emitting fluid from the ophthalmic device. The pore 940 may also be useful for connecting an end of the Micro fluidic Analytical System to its external region in an eye environment, which can allow for continuous monitoring without the removal of the ophthalmic device. In other embodiments, the pore 940 and pore control element 945 may be useful for flow control through the Micro fluidic Analytical System in a storage location, such as the fluid retention vessel 930. For example, while in storage, the Microfluidic Analytical System may be cleansed or refreshed by the flowing of solutions through the system and, in some embodiments, subjected to calibration protocols. Control of these functions may be performed by the integrated circuit components within the lens which may also be in communication with external controlling systems.

Energized Ophthalmic Devices with Lab on a Chip Components

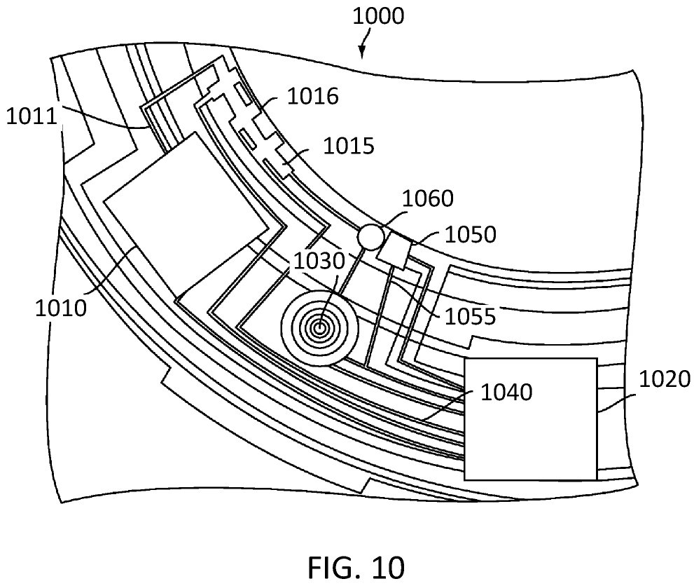

Referring now to FIG. 10, a top view section of an exemplary pumping mechanism 1000 for a Micro fluidic Analytical System using lab on a chip component 1010 is depicted. A lab on a chip component 1010 may share many aspects with the embodiment of the Microfluidic Analytical System that has been previously discussed. Similarly, however, in some embodiments small droplets may be moved around within the lab on a chip 1010 not through the action of a pump 1060 but by control of the droplets with EWOD components. Droplets may be combined in elements of the lab on a chip component 1010 to perform chemical processing. Numerous analysis techniques that may be performed, for example, in some embodiments the analysis of glucose as an analyte may be performed. The technique for this analysis may include, for example, an electrochemical or photometric technique as described or other techniques that may relate to the mixing of chemical substances that may be initially stored in the lab on a chip component 1010. Quantum-dot based spectroscopy techniques may be performed in or through the lab on a chip component. In some examples, QD based dyes may be located in a droplet that is mixed with a sample obtained from the environment of the biomedical device. The resulting interaction with a targeted analyte may yield spectroscopic signals that may be used to calculate a concentration of the analyte for example.

Various components such as energization elements (not shown), interconnects 1040, and sealing aspects previously described may take place in the annular Media Insert piece of the present example. Further, an electronic circuit 1020 capable of controlling various components including a lab on a chip component 1010 may be implemented. A pore 1050 and a pore control system 1055 may control the sampling of fluid samples from the ophthalmic device environment. A pump actuator 1030 may actuate a pump 1060 which may be mechanical in nature such as a membrane based pump. Droplets of a fluid sample may be pumped into micro-channel 1015 for metering of the volume and sample flow rate through the use of electrodes such as electrode 1016 as described in the present disclosure. The droplets may be provided to the lab on a chip component 1010 through a channel 1011 where it may be further processed. The lab on a chip component 1010 may use the pumped action on the sample to control flow within itself, or in other embodiments, it may control the flow rate of the sample provided to it on its own.

In additional embodiments, the lab on a chip component 1010 may be able to sense fluid in its environment without the need of an external pumping system. However, a pore 1050 may still be useful to provide control over flow of external fluid into the environment of the lab on a chip component. Thereafter the lab on a chip component 1010 may sample the introduced sample on its own, for example, by the control through electrowetting on dielectric or electrophoresis features that can attract and move fluid samples.

The lab on a chip component 1010 may comprise a design that may be consistent with the present description including, for example, very thin lab on chip flexible components to allow for the deformation into a shape consistent with the three dimensional shape of an ocular surface. In some embodiments, the shape and thickness of the lab on a chip component 1010 may allow it to be included in a planar form within the ophthalmic insert device.

Biomedical Devices with Quantum-Dot Spectrometers

FIG. 11A illustrates an exemplary QD Spectrometer system in a biomedical device 1100. The illustration in FIG. 11A may utilize a microfluidic system as illustrated in FIG. 10, or alternatively, may utilize a more passive approach to collecting samples wherein a sample fluid passively enters a channel 1102. The channel 1102 may be internal to the biomedical device in some examples and in other examples, as illustrated; the biomedical device may surround an external region with a reentrant cavity. In some examples where the biomedical device creates a channel of fluid external to itself, the device may also contain a pore 1160 to emit reagents or dyes to interact with the external fluid in the channel region. In a non-limiting sense, the passive sampling may be understood with reference to an example where the biomedical device may be a swallowable pill. The pill may contain regions that emit medicament 1150 as well as regions that analyze surrounding fluid such as gastric fluid for the presence of an analyte, where the analyte may be the medicament for example. The pill may contain controller 1170 regions proximate to the medicament where control of the release of the medicament may be made by portions of the biomedical pill device. An analysis region may comprise a reentrant channel within the biomedical pill device that allows external fluid to passively flow in and out of the channel. When an analyte, for example in gastric fluid, diffuses or flows into the channel it becomes located between the analysis region as depicted in FIG. 11A.

Referring now to FIG. 11B, once an analyte diffuses or otherwise enters the Quantum-dot spectrometer channel which shall be referred to as the channel 1102, a sample 1130 may pass in the emission portion of a Quantum-dot (QD) emitter 1110. The QD emitters 1110 may receive information from a QD emitter controller 1112 instructing the QD emitters 1110 to emit an output spectrum of light across the channel 1102.

In some examples, the QD emitter may act based on emission properties of the quantum-dots. In other examples, the QD emitter may act based on the absorption properties of the quantum-dots. In the examples utilizing the emission properties of the quantum-dots, these emissions may be photostimulated or electrically stimulated. In some examples of photostimulation; energetic light in the violet to ultraviolet may be emitted by a light source and absorbed in the quantum-dots. The excitation in the QD may relax by emitting photons of characteristic energies in a narrow band. As mentioned previously, the QDs may be engineered for the emission to occur at selected frequencies of interest. In a similar set of examples, QDs may be formed into the layered sandwiched mentioned previously between electrically active layers that may donate electrons and holes into the QDs. These excitations may similarly emit characteristic photons of selected frequency. The QD emitter 1110 may be formed by inclusion of nanoscopic crystals, that function as the quantum-dots, where the crystals may be controlled in their growth and material that are used to form them before they are included upon the emitter element.

In an alternative set of examples, where the QDs act in an absorption mode a combination of a set of filters may be used to determine a spectral response in a region. This mechanism is described in a prior section in reference to FIG. 2. Combinations of QD absorption elements may be used in analysis to select regions of the spectrum for analysis.

In either of these types of emission examples, a spectrum of light frequencies may be emitted by QD emitter 1110 and may pass thru the sample 1130. The sample 1130 may absorb light from some of the emitted frequencies if a chemical constituent within the sample is capable of absorbing these frequencies. The remaining frequencies that are not absorbed may continue on to the detector element, where QD receivers 1120 may absorb the photons and convert them to electrical signals. These electrical signals may be converted to digital information by a QD detector sensor 1122. In some examples the sensor 1122 may be connected to each of the QD receivers 1120, or in other examples the electrical signals may be routed to centralized electrical circuits for the sensing. The digital data may be used in analyzing the sample 1130 based on pre-determined values for QD wavelength absorbance values.

In FIG. 11C, the QD system is depicted in a manner where the sample is passed in front of spectral analysis elements that are spatially located. This may be accomplished for example in the manners described for the microfluidic progression. In other examples, the sample 1130 may contain analytes that diffuse inside an region of a biomedical device that encloses external fluid with material of the biomedical device to form a pore or cavity into which the sample may passively flow or diffuse to an analytical region that passes light from emitters within the biomedical device, outside the biomedical device, and again to detectors within the biomedical device. FIGS. 11B and 11C depict such movement as the difference between the locations of the sample 1130 which has moved along the analysis region to the new location 1131. In other examples the QDs may be consolidated to act in a single multidot location where the excitation means and the sensing means are consolidated into single elements for each function. Some biomedical devices such as ophthalmic devices may have space limitations for a spectrometer comprising more than a hundred quantum-dot devices, but other biomedical devices may have hundreds of quantum-dot devices which allow for a full spectrographic characterization of analyte containing mixtures.

The QD analytical system may also function with microfluidic devices to react samples containing analytes with reagents containing dyes. The dye molecules may react with specific analytes. As mentioned previously, an example of such a binding may be the FRET indicators. The dye molecules may have absorption bands in the ultraviolet and visible spectrum that are significantly strong, which may also be referred to as having high extinction coefficients. Therefore, small amounts of a particular analyte may be selectively bound to molecules that absorb significantly at a spectral frequency, which may be focused on by the QD analytical system. The enhanced signal of the dye complex may allow for more precise quantification of analyte concentration.

In some examples, a microfluidic processing system may mix an analyte sample with a reagent comprising a dye that will bind to a target analyte. The microfluidic processing system may mix the two samples together for a period that would ensure sufficient complexing between the dye and the analyte. Thereafter, in some examples, the microfluidic processing system may move the mixed liquid sample to a location containing a surface that may bind to any uncomplexed dye molecules. When the microfluidic system then further moves the sample mixture into an analysis region, the remaining dye molecules will be correlatable to the concentration of the analyte in the sample. The mixture may be moved in front of either quantum-dot emission light sources or quantum-dot absorption filters in the manners described.

A type of fluorescent dye may be formed by complexing quantum-dots with quenching molecules. A reagent mixture of quantum-dots with complexed quenching molecules may be introduced into a sample containing analytes, for example in a microfluidic cell, within a biomedical device. The quenching molecules may contain regions that may bind to analytes selectively and in so doing may separate the quenching molecule from the quantum-dot. The uncomplexed quantum-dot may now fluoresce in the presence of excitation radiation. In some examples, combinations of quantum-dot filters may be used to create an ability to detect the presence of enhanced emission at wavelengths characteristic of the uncomplexed quantum-dot. In other examples, other manners of detecting the enhanced emission of the uncomplexed quantum-dots may be utilized. A solution of complexed quantum-dots may be stored within a microfluidic processing cell of a biomedical device and may be used to detect the presence of analytes from a user in samples that are introduced into the biomedical device.

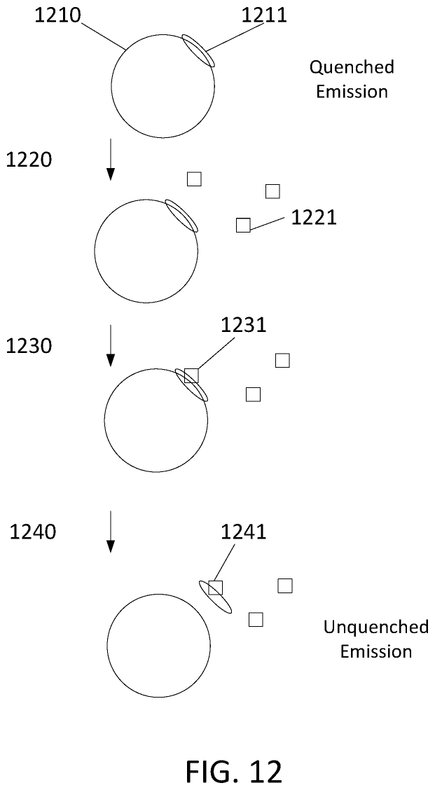

Referring to FIG. 12, an exemplary illustration of the concept of complexed quantum-dots acting as a dye is illustrated. A quantum-dot 1210 may comprise an exemplary material such as indium phosphide/zinc sulfide, copper indium sulfide/zinc sulfide, cadmium selenide, cadmium sulfide, lead sulfide, lead selenide, indium arsenide, and indium phosphide as examples. Other examples may comprise nanoparticles of silicon and carbon. Any material that may form a strained band structure of the type characteristic of a quantum-dot may be used in some examples. The quantum-dot core may be surrounded by a core shell coating that provides interface from the quantum-dot to its outside environment. For some examples, a biocompatible lipid coating which may allow for the binding of quenching molecules to the surface of the dot may also be provided. The quenching molecules 1211 may be bound to the quantum-dot surface and may act to facilitate electronic energy transfer from the quantum-dot which may result in a deexitation of the quantum-dot energy without fluorescent emission. A solution of the quantum-dots may be mixed 1220 with a sample containing analytes 1221. During the mixing, analytes may complex 1230 with the quenching molecules forming an analyte/quenching molecule complex 1231. The complexing of the analyte with the quenching molecule may decouple 1240 the quenching molecule from the quantum-dot, resulting in a free analyte/quenching molecule 1241 and an uncomplexed quantum-dot. Now, the quantum-dot may be excited by photons at energy distinct from the inherent fluorescent energy of the quantum-dot and the unquenched quantum-dot will now fluoresce. The concentration of the analyte in the sample may be a function of the fluorescence signal emanating from the unquenched quantum-dot. The microfluidic analysis system may include a light source, which may be a quantum-dot light emitting diode for example or other light sources with energy distinct from the fluorescence signal. A detector may be configured to detect all light that traverses a spectral analysis region. Alternatively, quantum-dot absorbance filters or other light filters may be used to selectively pass the energy band of the quantum-dot fluorescence signal.

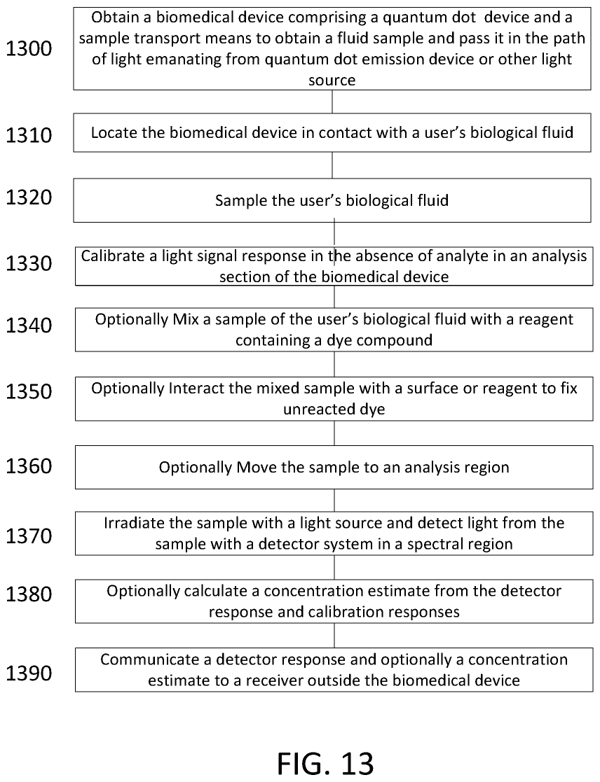

Referring to FIG. 13, an illustration of a flow diagram for analyte analysis in quantum-dot configured biomedical devices is provided. At step 1300, a user may obtain a biomedical device comprising: a quantum-dot device or reagent, and a sample transport mean. The biomedical device may be capable of obtaining a fluid sample from the user and pass it into the path of light emanating from a quantum-dot emission device or other light source. The light source may include a quantum-dot light emitting diode or a set of quenched quantum-dot filters configured to isolate selected spectral regions for analysis. Other light sources such as light emitting diodes and lasers may also be used. In some examples, the biomedical device may be obtained by an intermediary for the purposes of use by an end user. At step 1310, the biomedical device may be located in contact with a user's biological fluid. The location may include regions proximate to fluid emanations from a user such as, in non-limiting examples, tear fluid, blood, saliva, and waste products. Or, the location may include subcutaneous locations and locations within or in contact with the user's body cavity and venous system. At step 1320, the biomedical device is used to sample the user's biological fluid. At step 1330, the biomedical device may engage a calibration protocol either in an autonomous fashion or under direction of an external device or communication signal. The calibration may test the biomedical device's sample analysis section in the absence of an analyte to allow for a reference control signal that may be used in calculations related to an ultimate sample analysis signal. At step 1340, in some examples, an aliquot of the sample of user's biological fluid may be mixed with a reagent comprising a dye compound. The mixing may occur by passive diffusion based interaction, or alternatively, may be actively controlled such as with a microfluidic processing system as has been described herein. The dye compound may be an organic dye or, in some examples, a quantum-dot based dye. The dye may change a spectral characteristic, such as fluorescence emission or spectral absorbance, when it binds with an analyte in the sample. At step 1350, in some examples, the mixture may subsequently be processed to remove unreacted dye, particularly in examples where the binding of the dye to the analyte does not change a spectral characteristic of the dye. The sample may be mixed with a reagent that renders unbound dye inert or alternatively the sample may be passed in contact with a surface that may bind and immobilize or separate out unbound dye. At step 1360, the sample mixture may be moved from a reaction region to an analysis region in some examples. In other examples, the same location where reactions occur may be used to perform a spectral analysis. At step 1370, the sample may be irradiated with a light source. The light source may be from numerous example types including sources comprising quantum-dots as have been described. The irradiation may proceed through the sample and light that emerges from the sample may be detected in a spectral region by a detector system in the biomedical device. The detected emanation signal may be converted into an electrical signal and may also be converted into a digital data value that may also be conveyed as an electrical signal stream. At 1380, in some examples, the biomedical device may include on board processing devices and software algorithms that may allow for a calculation of an estimate of a concentration of the analyte in the sample. In other examples, the raw data signals, detector (calibration) signal without analyte, and detector signal with analyte may be transmitted without further signal processing in the biomedical device. At step 1390 the raw data signals may be communicated, for example, via wireless communication, to an external transceiver. In some examples, at step 1390, a calculated estimate of the concentration of an analyte may also be communicated. In still further examples, there may be numerous other sensor data that may be transmitted in addition to the analysis system data, which may include in a non-limiting perspective sensor measurements of the temperature sensed in a region of the biomedical device.

The QD spectrometer systems may be utilized in several different biomedical devices including: ophthalmic devices, biomedical pills, hygiene products, patches, and other similar biomedical products that are on or in proximity to the body in such a manner to detect and analyze analytes. The information obtained from the QD spectrometer system may be utilized for biometric analysis such as real-time readings of glucose for diabetics, as a non-limiting example. The information obtained may be communicated to a tertiary device, such as a smart phone, as disclosed in relationship to FIG. 4.

### Methods for Monitoring Bioanalytes

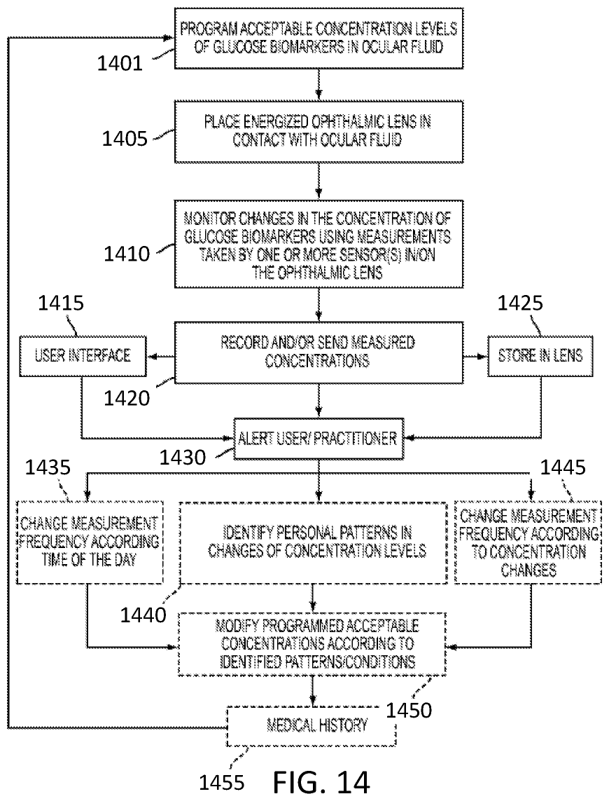

Referring now to FIG. 14, exemplary method steps that may be used to monitor analyte levels of a user wearing the ophthalmic lens according to aspects of the present disclosure are illustrated. At step 1401, thresholds values may be programmed into a software program. According to aspects of the present invention, threshold values may include, for example, acceptable levels for the concentration of glucose biomarkers in ocular fluid. The use of other biomarkers used to monitor different conditions such as depression, high blood pressure, and the like, are also within the inventive scope of aspects of the present disclosure. In addition, the preprogrammed levels may be different depending on whether the ocular fluid sample target is, for example, tear fluid or an interstitial fluid. The program may be stored and executed using one or both a processor forming part of the Media Insert of the ophthalmic device and an exterior device in communication with the processor of the Media Insert. An exterior device may include a smart phone device, a PC, a specialized biomedical device user interface, and the like; and may be configured to include executable code useful to monitor properties of ocular fluid samples. Ocular fluid properties may be measured by one or more sensors contained in the ophthalmic device. Sensors may include electrochemical sensors and/or photometric sensors. In an exemplary embodiment, the sensor analysis step may relate to a photometric sensing of glucose concentration based on a QD spectrometry. In another example, the sensor may detect the presence of reaction products from a glucose oxidase interaction with portions of the analyte sensor and the fluid sample.

At step 1405, the ophthalmic device including a microfluidic system may be placed in contact with a portion of the anterior ocular surface of the eye and worn by a user. In some embodiments, the ophthalmic device may be in a form of an energized contact lens and the step may be achieved when the contact lens is placed on the eye surface. In other embodiments, the biomedical device may be, for example, in the form of an intraocular lens, punctal plug, biomedical pill, or any other similar biomedical device, and still include aspects of the QD spectrometer system described in the present disclosure. Although the ophthalmic device is described throughout the specification in singular form, it will be understood by one skilled in the art that two ophthalmic devices (e.g. contact lenses), one placed on each eye, may function together to provide functionality aspects of the present disclosure.

At step 1410, concentration changes of biomarkers may be monitored using the one or more sensors. The monitoring of the biomarkers may occur at a predetermined frequency/bandwidth or upon demand through a user interface and/or an activation sensor in the ophthalmic device. Biomarkers can include those correlated to glucose levels, depression, blood pressure and the like. At step 1420, the processor of the ophthalmic device can record the measured property/condition from a sample of ocular fluid. In some embodiments, the processor of the ophthalmic device may store it and/or send it to one or more device(s) in communication with the ophthalmic device. At step 1415, the value recorded can be stored and analyzed in the user interface in communication with the ophthalmic lens, and/or, at step 1425, the analysis and recording can take place in the ophthalmic device.

At step 1430, one or both the ophthalmic device and the user interface may alert the user, and/or a practitioner, of the measured concentration. The alert may be programmed to occur when the levels measured are outside the predetermined threshold values programmed, received and/or calculated by the ophthalmic device. In addition in some embodiments, the data and alerts may be analyzed to perform one or more steps of: a) change measurement frequency according to the time of the day, b) identify personal patters in the changes of concentration levels measures, and c) change the measurement frequency according to the changes in concentrations measured. At step 1435, the time of the day may change the frequency of measurements. For example, if the ophthalmic device is one that would remain in the eye during sleep, the number of measurements during 10 pm and 6 am can decrease or stop. Similarly, during lunch and dinner times the frequency may increase to detect changes due to the food consumption of the user. At step 1440, patterns in changes of the concentration levels may be identified by the system. Using the identified patterns, the system may alert the user of causes and/or, at step 1445, change the frequency according to the identified changes so that the system is more alert during critical identified conditions. Critical conditions can include events that would trigger a significant increase or decrease in glucose levels. Events can include, for example, holiday dates, exercise, location, time of the day, consumption of medicaments and the like.

In some embodiments, at step 1450, the originally programmed values may be customized, periodically or in real time, according to identified patterns/conditions. This ability may allow the system to increase its effectiveness by eliminating false alarms and increasing sensitivity at a critical condition. Effectiveness can promote user participation with the system thereby maximizing the benefits of the ophthalmic device and thereby providing a safe monitoring system. At step 1455, data relating to the user including, for example, the identified patterns, measurements, and/or preferences may become part of the medical history of the user. Medical history may be stored securely by encrypting the data and/or restricting its access.

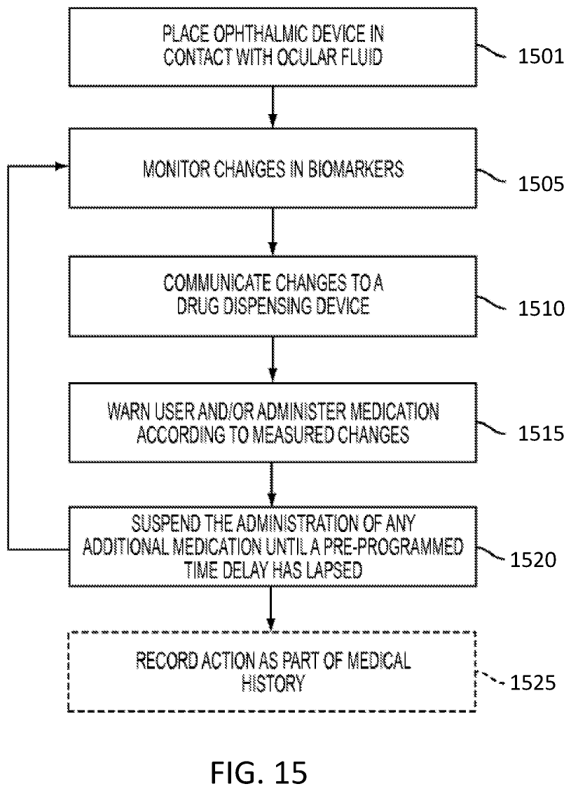

Referring now to FIG. 15, exemplary method steps that may be used to treat the glucose levels of a user wearing the ophthalmic lens according to aspects of the present disclosure are illustrated. At step 1501, an ophthalmic device including a QD spectrometer analytical system is placed in contact with ocular fluid. In some embodiments, the ophthalmic device may be in a form of an energized contact lens and the step may be achieved when the contact lens is placed on the eye surface. In other embodiments, the ophthalmic device may be, for example, in the form of an intraocular lens or a punctal plug, and still include aspects of the QD spectrometer system described in the present disclosure.

At step 1505, changes in biomarkers in the ocular fluid can be monitored. Methods of monitoring the biomarker changes may include, for example, steps illustrated in FIG. 14. At step 1510, measured changes can be communicated in real time to a medicament-dispensing device in direct or indirect communication with the ophthalmic device. Although the changes in concentration of the monitored biomarkers in ocular fluid may include a time delay in relation to the concentration changes in the bloodstream of the user, upon detection, at step 1515 the medicament-dispensing device may administer a medicament capable of lowering or raising concentrations to a normal level. For example, glucose levels may be monitored and treated when they are outside a normal level. Continuous monitoring may prevent uncontrolled blood sugar levels which may damage the vessels that supply blood to important organs, like the heart, kidneys, eyes, and nerves. Because an individual whose glucose levels may reach a level that exposes him/her to the risks may feel fine, aspects of the present disclosure may help take action upon early detection of the condition. Early detection may not only bring back levels to a normal condition and/or make the user aware, but additionally prevent the more dramatic and permanent consequences including, for example, a heart attack or stroke, kidney failure, and blindness which have been known to occur when abnormal glucose levels are left untreated.

In addition, in some embodiments the medicament administering device may send an alert to the user through its interface or using component of the ophthalmic device. For example, in some ophthalmic device embodiments the Media Insert may include a light projection system, such as one or more LEDs, capable of sending a signal to the user.

Subsequently at step 1520, any further drug administering can be suspended to prevent overdosing of the system due to the time delay of the effect of the drug and the effect to be reflected in the tear fluid. For example, the medicament may require 10-30 minutes to counteract the abnormal level, and upon its effect, may take another 20 minutes to equalize concentrations in tear fluid. Consequently, programmed algorithms capable of correlating the condition, time delay, and appropriate subsequent dosing of medicaments can be programmed in the system to function safely. At step 1525, data relating to one or both the measured conditions and the medicament administration to the user may be stored and used as part of a treatment and/or medical history of the user.

Specific examples have been described to illustrate sample embodiments for the methods and devices related to inclusion of quantum-dots for spectroscopic analysis in biomedical devices. These examples are for said illustration and are not intended to limit the scope of the claims in any manner. Accordingly, the description is intended to embrace all examples that may be apparent to those skilled in the art.

What is claimed is:

1. A biomedical device comprising:
an energization element;
an external encapsulation boundary, wherein at least a portion of the boundary comprises an electrically controlled pore operative to allow a fluid sample to pass into the biomedical device from an external region;
a microfluidic processing chip operative to mix the fluid sample with a reagent comprising an analyte specific dye;
a quantum-dot light emitter installed to emit light through a portion of the microfluidic processing chip; and
a photodetector installed on a distal side of the microfluidic processing chip from the quantum-dot light emitter, wherein light emitted by the quantum-dot light emitter proceeds through a top surface of the microfluidic processing chip, through a sample analysis region of the microfluidic processing chip, through a bottom surface of the microfluidic processing chip and into the photodetector;
a radio frequency transceiver; and
an analog-to-digital converter, wherein a signal from the photodetector is converted to a digital data value that is transmitted outside the biomedical device by the radio frequency transceiver.

2. The biomedical device of claim 1 further comprising a pump configured to facilitate the passage of the fluid sample into the medical device fluid from the external region via the electronically controlled pore.

3. The biomedical device of claim 2 wherein the pump comprises a flexible and collapsible membrane capable of activation upon an application of pressure upon the membrane.

4. The biomedical device of claim 3 wherein the application of pressure upon the membrane is produced by one or more of: a flow of a contained fluid between a cavity and a tube connecting the cavity to the pump; an expansion of the membrane caused by an application of voltage upon a piezoelectric component; or a electrowetting on dielectric device.

5. The biomedical device of claim 1 wherein the device is a contact lens.

6. The biomedical device of claim 1 wherein the device is an electronic pill.

7. The biomedical device of claim 6 wherein the electronic pill comprises a release mechanism controllable to release medicament based on the signal received at the photodetector.

8. The biomedical device of claim 6 wherein the electronic pill comprises a release mechanism controllable to release a quantum-dot dye into the microfluidic processing chip, wherein the dye reacts with analyte molecules and allows the quantum light emitter to excite the quantum-dot dye to emit light whose intensity is correlated to a concentration of the analyte molecules.

9. The biomedical device of claim 1 further comprising an electrode for measuring a flow rate of fluid.

10. The biomedical device of claim 1 further comprising a fluid retention vessel.

11. The biomedical device of claim 1 further comprising a pore control element configured to equalize gas pressure or to cause fluid to be emitted from the ophthalmic device.
